(19) United States
(12) Patent Application Publication
(10) Pub. No.: US 2015/0305309 A1
(43) Pub. Date: Oct. 29, 2015

(54) MULTIFUNCTIONAL PET DIAPER

(71) Applicant: JIANGSU ZHONGHENG PET ARTICLES JOINT-STOCK CO.,LTD, Jiangsu (CN)

(72) Inventor: Bin QIU, Jiangsu (CN)

(21) Appl. No.: 14/683,621

(22) Filed: Apr. 10, 2015

(30) Foreign Application Priority Data
Apr. 27, 2014 (CN) 201420229647.7

(51) Int. Cl. A01K 23/00; A61B 5/145; A61B 10/00

(52) U.S. Cl. CPC A01K 23/00; A61B 10/007; A61B 5/14532; A61B 5/14539

(57) ABSTRACT

A multifunctional pet diaper includes a surface layer, absorbing layer and anti-leak layer overlapping with each other. A middle section of the absorbing layer defines an absorbing area. A peripheral section of the surface layer and the anti-leak layer surrounds the absorbing area. An area of the anti-leak layer is larger than an area of the absorbing layer, and an area of the surface layer is larger than the area of the absorbing layer. The pet diaper further includes more than one detecting member in the absorbing area. The detecting members are configured to collect and detect urine and/or feces, and to directly show the test results by changing the color of the detecting members.

### CROSS REFERENCE TO RELATED APPLICATION

The present application is based on, and claims priority from, Chinese Application Number 201420229647.7 filed Apr. 27, 2014, the disclosure of which is incorporated herein in its entirety.

### TECHNICAL FIELD

The present disclosure relates to a pet product, in particular a multifunctional pet diaper.

### BACKGROUND

With the development of society and improvement in living standards, people are enjoying the material life, while being constantly in pursuit of the spiritual life, therefore, it has become fashionable to keep a pet. Pets are people's spiritual support, can provide company, and bring a lot of joy, therefore, pets have a more and more important position in people's lives, and people have increasingly higher requirements for the quality of life of pets. Pets have good living conditions but with sufficient food and lack of exercise they are prone to obesity and can have various complications, especially diabetes, which seriously affect the pet's quality of life and cause worry for pet owners. People need to take pets to have a health examination and treatment on a regular basis to ensure the health of pets, but it is neither convenient nor hygienic to manually take urine and feces for testing. In everyday life, a pet diaper has been extensively used for cleaning up pet excrements. A wide variety of pet diapers are now available, and most of them only have the leak-proof and anti-overflow functions and cannot test the urine and feces.

### BRIEF DESCRIPTION OF THE DRAWINGS

The present disclosure will be further described in conjunction with drawings and embodiments. Aspects of the present disclosure are to be understood from the following detailed description when read with the accompanying figures which are incorporated herein by reference.

FIG. 1 is a top view of a pet diaper in accordance with at least a first embodiment.

FIG. 2 is a cross-section view of the pet diaper along the line A-A of FIG. 1 in accordance with at least the first embodiment.

FIG. 3 is a top view of a pet diaper in accordance with at least a second embodiment.

FIG. 4 is a cross-section view of the pet diaper along the line B-B in accordance with at least the second embodiment.

### DETAILED DESCRIPTION

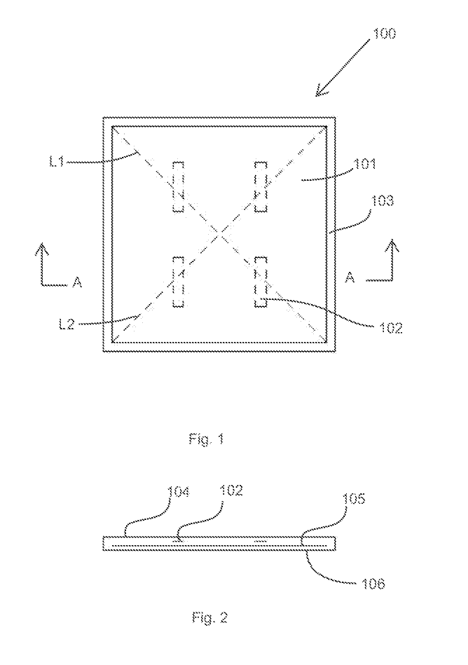

An embodiment, as described in connection with FIGS. 1-2, is a multifunctional pet absorbent article, e.g., a pet diaper 100. In at least one embodiment, the pet diaper 100 has a square shape as shown in the top view of FIG. 1. The shape of the pet diaper 100 is not limited to the square shape as shown in FIG. 1. Other shapes, e.g., a rectangular shape, can be suitable for the pet diaper in other embodiments.

The pet diaper 100 includes a middle section 101 defining an absorbing area 101 for absorbing urine and/or feces, a peripheral section 103 including edges of the pet diaper 100 and surrounding the middle section 101. In the pet diaper 100, one or more detecting members 102 are distributed in the urine absorbing area 101. In at least one embodiment, the detecting members 102 are urinary glucose test strips printed on a paper, e.g., toilet paper. The detecting members 102 are positioned closer to a center of the diaper than the edges of the pet diaper 100. The center of the diaper 100 as shown in FIG. 1 is defined as an intersection of two imaginary lines L1 and L2 each of which connects two opposing corners of the square shaped diaper 100. In at least some embodiments, detecting members 102 are positioned along at least one imaginary line connecting two opposing corners. In at least some embodiments, there are a number of detecting members 102 corresponding to twice the number of intersecting lines. In at least some embodiments, there are fewer detecting members 102 than two times the number of intersecting lines. In some embodiments, the detecting members 102 are embedded, printed, or laminated in the surface layer 104 or the absorbing layer 105.

FIG. 2 is a cross-section view along lines A-A of FIG. 1. As illustrated in FIG. 2, the pet diaper 100 includes a surface layer 104 as a top surface and an anti-leak layer 106 as a bottom layer. In at least one embodiment, the surface layer 104 is formed of non-woven fabrics and the absorbing layer 105 is formed of toilet paper.

The pet diaper 100 further includes an absorbing layer 105 sandwiched between the surface layer 104 and the anti-leak layer 106. The absorbing layer 105 defines the absorbing area 101 of the pet diaper 100 and does not extend to the peripheral section 103. An area of the anti-leak layer 106 is larger than an area of the absorbing layer 105, and an area of the surface layer 104 is larger than the area of the absorbing layer 105. The detecting members 102 are provided between the surface layer 104 and the absorbing layer 105.

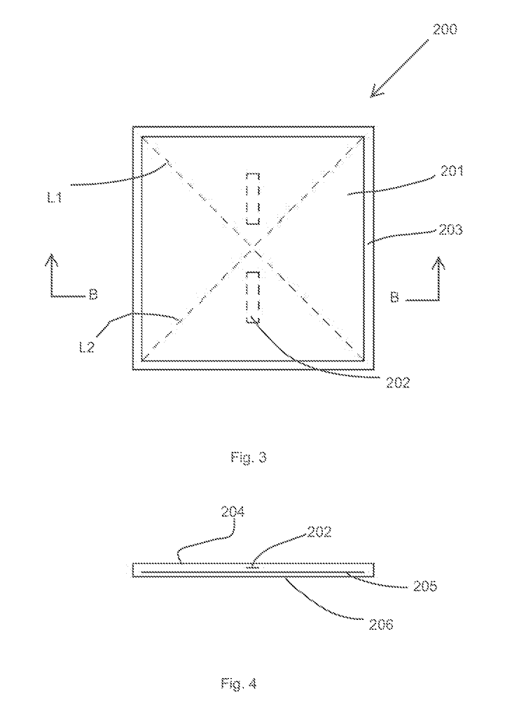

Another embodiment, as described in connection with FIGS. 3-4, is a multifunctional pet absorbent article, e.g., a pet diaper 200. The pet diaper 200 is similar to the pet diaper 100 as shown in FIGS. 1-2 except the position of the detecting members 202.

In at least one embodiment, the pet diaper 200 has a square shape as shown in the top view of FIG. 3. The shape of the pet diaper 200 is not limited to the square shape as shown in FIG. 3. Other shapes, e.g., a rectangular shape, can be suitable for the pet diaper in other embodiments. The pet diaper 200 includes a middle section 201 defining an absorbing area 201 for absorbing urine and/or feces, a peripheral section 203 including edges of the pet diaper 200 and surrounding the middle section 201. In the pet diaper 200, one or more detecting members 202 are distributed in the urine absorbing area 101. In at least one embodiment, the detecting members 202 are urinary glucose test strips printed on a paper, e.g., toilet paper. As shown in FIG. 3, a pair of detecting members 202 includes two urinary glucose test strips attached on a paper, e.g., toilet paper. The detecting members 202 have their center line passing through a center of the pet diaper 200. The center of the diaper 200 as shown in FIG. 3 is defined as an intersection of two imaginary lines L1 and L2 each of which connects two opposing corners of the square shaped diaper 200. In at least some embodiments, there is a corresponding detecting member 202 positioned between corresponding pairs of imaginary lines. In at least some embodiments, there are fewer detecting members 202 than two times the number of intersecting lines. In some embodiments, the detecting members 202 are embedded, printed, or laminated in the surface layer 204 or the absorbing layer 205.

FIG. 4 is a cross-section view along lines B-B of FIG. 3. As illustrated in FIG. 3, the pet diaper 200 includes a surface layer 204 as a top surface and an anti-leak layer 206 as a bottom layer. In at least one embodiment, the surface layer 204 is formed of non-woven fabrics and the absorbing layer 205 is formed of toilet paper.

The pet diaper 200 further includes an absorbing layer 205 sandwiched between the surface layer 204 and the anti-leak layer 206. The absorbing layer 205 defines the absorbing area 201 of the pet diaper 200 and does not extend to the peripheral section 203. An area of the anti-leak layer 206 is larger than an area of the absorbing layer 205, and an area of the surface layer 204 is larger than the area of the absorbing layer 205. The detecting members 202 are provided between the surface layer 204 and the absorbing layer 205.

According to the embodiments, the detecting members 102, 202 are configured to detect pet excrement while the absorbing area collects the pet excrement therefore the pet diaper can reveal the pet's physical condition based on the result or the change shown on the detecting members 102, 202. In some embodiments, the test strips are urine test strips used in a glycosuria test to check the sugar level of the urine. For example, if the pet develops diabetes, in the diaper with the provided glucose test strips, when the pet urinates, the urine flows into the absorbing layer through the surface layer and then is absorbed by the absorbing layer. Upon dipping, the reagent in the test strip reacts with the material in the urine to produce a new material which can present different colors based on different sugar levels of the material in the urine. Thus, according to the content of glucose in the urine, the detecting member presents the color of light blue, light green, brown or dark brown. Urine glucose levels are measured by colorimetric methods, so that the pet diet can be controlled in a timely manner, and decided decision can be made whether to seek medical treatment.

Other embodiments of the detecting members are also suitable for the pet diaper. For example, the detecting member can be a pH value test strip, a bacterial testing strip, a urine dipstick and/or a variety of other commercially available test strips.

In some embodiments, a multifunctional pet diaper includes a top layer, an absorbing layer, and a bottom layer. A middle section of the absorbing layer defines an absorbing area. A peripheral section of the top layer and the bottom layer surrounds the absorbing area. The multifunctional pet diaper further includes a detecting member located in the absorbing area and configured to detect urine and/or feces. The absorbing area includes the top layer, the absorbing layer and the bottom layer overlapping each other. An area of the bottom layer is larger than that of the absorbing layer, and an area of the top layer is larger than that of the absorbing layer.

The foregoing outlines features of several embodiments so that those skilled in the art may better understand the aspects of the present disclosure. Those skilled in the art should appreciate that they may readily use the present disclosure as a basis for designing or modifying other processes and structures for carrying out the same purposes and/or achieving the same advantages of the embodiments introduced herein. Those skilled in the art should also realize that such equivalent constructions do not depart from the spirit and scope of the present disclosure, and that they may make various changes, substitutions, and alterations herein without departing from the spirit and scope of the present disclosure.

What is claimed is:

1. A multifunctional pet diaper, comprising:
a top layer;
an absorbing layer;
a bottom layer;
a middle section of the absorbing layer defining an absorbing area, and a peripheral section of the top layer and the bottom layer surrounding the absorbing area; and
a detecting member located in the absorbing area and configured to detect urine and/or feces,
wherein the top layer, the absorbing layer and the bottom layer overlap each other in a thickness direction of the pet diaper, and
wherein an area of the bottom layer is larger than an area of the absorbing layer, and an area of the top layer is larger than the area of the absorbing layer.

2. The multifunctional pet diaper as claimed in claim 1, wherein said top surface is formed of non-woven fabrics, and the absorbing layer is formed of toilet paper.

3. The multifunctional pet diaper as claimed in claim 1, wherein said detecting member is embedded in the top surface.

4. The multifunctional pet diaper as claimed in claim 1, wherein said detecting member is printed on the top surface.

5. The multifunctional pet diaper as claimed in claim 1, wherein said detecting member is laminated with the top surface.

6. The multifunctional pet diaper as claimed in claim 1, wherein said detecting member is embedded in the absorbing layer.

7. The multifunctional pet diaper as claimed in claim 1, wherein said detecting member is printed on the absorbing layer.

8. The multifunctional pet diaper as claimed in claim 1, wherein said detecting member is laminated with the absorbing layer.

9. The multifunctional pet diaper as claimed in claim 2, wherein said detecting member is printed on the non-woven fabric.

10. The multifunctional pet diaper as claimed in claim 2, wherein said detecting member is printed on the toilet paper.

11. The multifunctional pet diaper as claimed in claim 1, wherein said detecting member includes urinary glucose test strips.

12. The multifunctional pet diaper as claimed in claim 1, wherein said detecting member includes pH value strips.

13. The multifunctional pet diaper as claimed in claim 1, wherein said detecting member includes bacterial testing strips.

14. The multifunctional pet diaper as claimed in claim 1, wherein said detecting member includes first and second detecting members, and a center line connecting centers of the first and second detecting members passes through a center of the diaper.

15. The multifunctional pet diaper as claimed in claim 1, wherein said detecting member includes four detecting members distributed close to a center of the diaper.

16. The multifunctional pet diaper as claimed in claim 2, wherein said detecting member includes first and second detecting members, and a center line connecting centers of the first and second detecting members passes through a center of the diaper.

17. The multifunctional pet diaper as claimed in claim 2, wherein said detecting member includes four detecting members distributed close to a center of the diaper.

18. The multifunctional pet diaper as claimed in claim 1, wherein
a center of the diaper is defined as an intersection of intersecting lines, each of the intersecting lines connects two opposing portions of the diaper, and a number of detecting members corresponds to twice the number of intersecting lines.

19. The multifunctional pet diaper as claimed in claim 1, wherein
a center of the diaper is defined as an intersection of intersecting lines,
each of the intersecting lines connects two opposing portions of the diaper, and
a number of the detecting members is fewer than two times the number of intersecting lines.
